(19) United States
(12) Patent Application Publication
(10) Pub. No.: US 2021/0251558 A1
(43) Pub. Date: Aug. 19, 2021

(54) SYSTEMS, METHODS, AND DEVICES FOR DETERMINING MILD TRAUMATIC BRAIN INJURIES

(71) Applicant: Research Triangle Institute, Research Triangle Park, NC (US)

(72) Inventors: Kristin Hedgepath Gilchrist, Rockville, MD (US); Sonia Grego, Durham, NC (US); Gregory Frederick Lewis, Bloomington, IN (US); Meghan Sarah Hegarty-Craver, Durham, NC (US)

(21) Appl. No.: 17/236,266

(22) Filed: Apr. 21, 2021

Related U.S. Application Data
(63) Continuation of application No. PCT/US19/57518, filed on Oct. 23, 2019.
(60) Provisional application No. 62/749,716, filed on Oct. 24, 2018.

(51) Int. Cl. A61B 5/00; A61B 5/352; A61B 5/11; A61B 5/0205; G16H 50/30

(52) U.S. Cl. CPC A61B 5/4064; A61B 5/352; A61B 5/1124; A61B 5/0205; A61B 5/7275; A61B 5/02416; A61B 5/002; A61B 5/4884; G16H 50/30; A61B 2562/0219; A61B 2503/10; A61B 5/0006

(57) ABSTRACT

A computer implemented method is disclosed for detecting a mild traumatic brain injury (mTBI) on a subject having recently suffered a head injury. The method includes (1) receiving first electrocardiogram (ECG) data from the subject during a first transition from a first position to a second position; and (2) determining a probability value of mTBI within the subject based at least partially on the first ECG data during the first transition. The first position may be a prone position or a supine position and the second position may be a standing position.

### CROSS-REFERENCE TO RELATED APPLICATION

This application is a continuation of International patent application no. PCT/US2019/057518 titled “SYSTEMS, METHODS, AND DEVICES FOR DETERMINING MILD TRAUMATIC BRAIN INJURIES” filed on Oct. 23, 2019, which claims the benefit of priority of U.S. provisional patent application No. 62/749,716 titled “Systems, Methods, and Devices for Determining Mild Traumatic Brain Injuries” filed on Oct. 24, 2018, which are incorporated herein in their entireties by this reference.

### STATEMENT REGARDING FEDERALLY SPONSORED RESEARCH OR DEVELOPMENT

This invention was made with government support under Contract No. W911QY-16-C-0141 awarded by the Telemedicine and Advanced Technology Research Center (TATRC) at the United States Army Medical Research and Materiel Command (USAMRMC) through the AMEDD Advanced Medical Technology Initiative (AAMT). The government has certain rights in the invention.

### FIELD

The present disclosure relates generally to field based diagnostic medical devices. More specifically; methods, systems, and devices are disclosed for objectively diagnosing a possible mild traumatic brain injury (mTBI) on location of the injury.

### BACKGROUND

There is rising awareness of the widespread nature and long term damage caused by a mild traumatic brain injury (mTBI), also called a concussion. Headaches, fatigue, insomnia, dizziness, irritability, balance issues, cognitive impairment, and other emotional problems may result from mTBI and may be diagnosed as post-concussion syndrome (PCS). Normal activities may worsen the symptoms of PCS and cause patients to have to restructure their lives. For children this may be limited participation in sports and other extra-curricular activities. Extended absences from school, due to PCS, may even cause a child to have to repeat a grade. Adults suffering from PCS may have difficulty focusing, communicating, and/or otherwise being effective at work. Other impacts include disruptions of personal life and family life.

Many sports such as football, soccer, rugby, basketball, baseball, and martial arts are known to place participants at risk of mTBI. Additionally, mTBI has been one of the most common injuries in recent military combat operations. Other sources of mTBI can be falls and domestic abuse (including violent shaking). The long-term damage of mTBI may be mitigated if appropriate care is given within one hour of the injury (i.e. the so-called “golden hour”). However, the diagnosis for mTBI within the “golden hour” is difficult. Many times the signs and symptoms can be very subtle. Additionally, the one suffering the head injury may be reluctant or refuse to seek medical attention at a clinic, hospital, or other emergency care location.

Accordingly, there remains a need for systems, methods, and devices of objectively diagnosing mTBI amenable to low-cost operation in the field. Additionally, the methods and devices should require minimal training by a responsible individual (e.g. coach, parent, commanding officer, first responder, etc.) administering the diagnosis.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A computer implemented method is disclosed for detecting a mild traumatic brain injury (mTBI) on a subject having recently suffered a head injury. The computer implemented method includes (1) receiving first beat to beat heart interval data from the subject during a first transition from a first position to a second position; and (2) determining a probability value of mTBI within the subject based at least partially on the heart interval data during the first transition. The first position may be a prone position or a supine position and the second position may be a standing position.

In some embodiments, the first heart interval data may be received in near real-time from monitoring of an electrocardiogram (ECG) or other heart rate sensor positioned on the subject. The method may further include receiving first accelerometer data from an accelerometer positioned on the subject and the first accelerometer data may be three-axis accelerometer data. The method may further include determining the first transition based at least partially on the first accelerometer data. The method may also include receiving second heart interval data during a second transition of the subject from a sitting position to a standing position.

In some embodiments, the method may further include determining a first baroreflex response associated with the first transition and/or determining a second baroreflex response associated with the second transition. Determining the first baroreflex response may include determining a magnitude, a rate of change, and/or a recovery time of beat-to-beat (RR) intervals within the first ECG data. Determining the second baroreflex response may include determining a magnitude, a rate of change, and/or a recovery time of RR intervals within second ECG data.

In some embodiments, the computing device may be configured to wirelessly receive the first ECG data and the second ECG data over a personal area network (PAN) and the PAN may be a Bluetooth network or the like. In other embodiments, the computing device may be configured to wirelessly receive the first ECG data and the second ECG data over a local area network (LAN) and the LAN may be a Wi-Fi network or the like. In still other embodiments, the computing device may be configured to wirelessly receive the first ECG data and the second ECG data over a wide area network (WAN) and the WAN may be a 3G network, a 4G network, a 5G network, or the like.

In some embodiments, the computing device may be a laptop, a tablet, a smartphone, a smartwatch, or the like. In other embodiments, the computing device may be a server, a personal computer (PC), or the like. In still other embodiments, the computing device may be implemented within a medical device. The subject may be a human subject and the human subject may be an athlete having recently received a head injury associated with participation in a sport. The sport may be football, soccer, rugby, basketball, baseball, martial arts, or the like. The first ECG data and the second ECG data may be received from a chest positioned ECG sensor, an ear positioned ECG sensor, or the like.

In another embodiment, a computing device is disclosed for detecting an mTBI on a subject having recently suffered a head injury. The computing device includes a memory and at least one processor configured for performing a method. The method includes (1) receiving first electrocardiogram (ECG) data from the subject during a first transition from a first position to a second position; and (2) determining a probability value of mTBI within the subject based at least partially on the first ECG data during the first transition. The first position may be a prone position or a supine position, and the second position may be a standing position.

In another embodiment, a non-transitory computer-readable storage medium is disclosed. The non-transitory computer-readable storage medium is configured for storing instructions to be implemented on a computing device including at least one processor, the instructions when executed by the at least one processor cause the at least one computing device to perform a method for detecting an mTBI on a subject having recently suffered a head injury. The method includes (1) receiving first electrocardiogram (ECG) data from the subject during a first transition from a first position to a second position; and (2) determining a probability value of mTBI within the subject based at least partially on the first ECG data during the first transition. The first position may be a prone position or a supine position, and the second position may be a standing position.

In another embodiment, a computer implemented method is disclosed for detecting an mTBI on a subject having recently suffered a head injury. The computer implemented method includes (1) receiving first beat-to-beat heart interval data from a sensor configured to monitor the subject during a first transition from a first position to a second position; and (2) determining a probability value of mTBI within the subject based at least partially on the first beat-to-beat heart interval data during the first transition.

In some embodiments, the sensor may be an electrocardiography (ECG) sensor, a photoplethsmography (PPG) sensor, an ultrasonic sensor, a pressure sensor, a sensor positioned on the ear, or the like. In other embodiments, the sensor may be any type of sensor configured to receive heart rate data.

In some embodiments, the computing device may be implemented within a diagnostic device that includes the sensor. In other embodiments, the computing device may be implemented within a mobile device configured for receiving the first beat-to-beat heart interval data over a personal area network (PAN) interface and/or a local area network (LAN) interface. In still other embodiments, the computing device may be implemented within a remote server configured for receiving the first beat-to-beat heart interval data over a wide area network (WAN).

In another embodiment, a computing device is disclosed for detecting an mTBI on a subject having recently suffered a head injury. The computing device includes a memory and at least one processor configured for performing a method. The method includes (1) receiving first beat-to-beat heart interval data from a sensor configured to monitor the subject during a first transition from a first position to a second position; and (2) determining a probability value of mTBI within the subject based at least partially on the first beat-to-beat heart interval data during the first transition. The first position may be a prone position or a supine position and the second position may be a standing position.

In another embodiment, a non-transitory computer-readable storage medium is disclosed. The non-transitory computer-readable storage medium is configured for storing instructions to be implemented on a computing device including at least one processor, the instructions when executed by the at least one processor cause the at least one computing device to perform a method for detecting an mTBI on a subject having recently suffered a head injury. The method includes (1) receiving first beat-to-beat heart interval data from a sensor configured to monitor the subject during a first transition from a first position to a second position; and (2) determining a probability value of mTBI within the subject based at least partially on the first beat-to-beat heart interval data during the first transition.

In another embodiment, a computer implemented method is disclosed for detecting an mTBI on a subject having recently suffered a head injury. The computer implemented method includes (1) receiving first beat-to-beat heart interval data from a sensor configured to monitor the subject during a baroreflex response within the subject; and (2) determining a probability value of mTBI within the subject based at least partially on the first beat-to-beat heart interval data during the baroreflex response.

In some embodiments, a Valsalva maneuver or a cold pressor test may be used to produce the baroreflex response of the subject.

In another embodiment, a computing device is disclosed for detecting an mTBI on a subject having recently suffered a head injury. The computing device includes a memory and at least one processor configured for performing a method. The method includes (1) receiving first beat-to-beat heart interval data from a sensor configured to monitor the subject during a baroreflex response within the subject; and (2) determining a probability value of mTBI within the subject based at least partially on the first beat-to-beat heart interval data during the baroreflex response.

In another embodiment, a non-transitory computer-readable storage medium is disclosed. The non-transitory computer-readable storage medium is configured for storing instructions to be implemented on a computing device including at least one processor, the instructions when executed by the at least one processor cause the at least one computing device to perform a method for detecting an mTBI on a subject having recently suffered a head injury. The method includes (1) receiving first beat-to-beat heart interval data from a sensor configured to monitor the subject during a baroreflex response within the subject; and (2) determining a probability value of mTBI within the subject based at least partially on the first beat-to-beat heart interval data during the baroreflex response.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE FIGURES

The present embodiments are illustrated by way of example and are not intended to be limited by the figures of the accompanying drawings. In the drawings:

FIG. 1 depicts a block diagram illustrating a system including an electrocardiogram (ECG) device including an ECG sensor and an accelerometer, a computing device, and analysis software (operating within the computing device) for field diagnosis of mild traumatic brain injury (mTBI) in accordance with embodiments of the present disclosure.

FIG. 2 depicts a diagram illustrating three sequences (i.e. methods) using the system of FIG. 1 for providing an objective diagnosis of mTBI in the field in accordance with embodiments of the present disclosure.

FIG. 3 depicts a graph illustrating beat-to-beat (RR) intervals versus time of a test subject before during and after a transition from a first position to a second position utilizing a low pass filtering technique in accordance with embodiments of the present disclosure.

FIG. 4 depicts another graph illustrating RR intervals versus time of a test subject utilizing an alternate low pass filtering in accordance with embodiments of the present disclosure.

FIG. 5 depicts a graph illustrating a true positive rate versus a false positive rate of a combined model taken from sequence 1 data and sequence 2 data of the diagram of FIG. 3 in accordance with embodiments of the present disclosure.

FIG. 6 depicts a block diagram illustrating a system using a smart tablet for objective diagnosis mTBI using a sequence of a test subject laying down and then sitting up in accordance with embodiments of the present disclosure

FIG. 7 depicts a block diagram illustrating a smart tablet that provides the computing device of FIG. 1 and/or the smart tablet of FIG. 6 in accordance with embodiments of the present disclosure.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description. References to “one embodiment” or “an embodiment” in the present disclosure can be, but not necessarily are, references to the same embodiment and such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Disclosed herein are systems, methods, and devices for objectively diagnosing mTBI of a subject recently experiencing a head injury amenable to low-cost operation in the field. Additionally, the systems, methods, and devices require minimal training by a responsible individual (e.g. coach, teammate, parent, commanding officer, first responder, co-worker, etc.) administering the diagnosis.

The disclosed approach takes advantage of short-term cardiovascular responses to postural changes that are regulated by the autonomic nervous system (ANS). Autonomic dysfunction associated with mTBI may be reflected in these cardiovascular responses and may be assessed with a non-invasive heart monitor. Short-term cardiovascular regulation of blood flow during posture changes, a process largely coordinated by the (ANS), is dysregulated in orthostatic disorders. There is evidence of altered cardiovascular regulation after acute traumatic brain injury (TBI). Acute TBI of mild and moderate severity is associated with an early significant modification in arterial baroreflex sensitivity in an animal model and a retrospective study of concussed subjects suggests that post-concussion dizziness may reflect more orthostatic intolerance rather than vestibular impairment in injured subjects.

This approach provides a rapid assessment of brain injury (e.g. mTBI) based on impaired feedback between the central nervous system and a peripheral organ, specifically arterial baroreflex sensitivity. Physiological measurements are acquired by non-invasive wearable sensors and an algorithm provides a parameter (e.g. probability) describing normal or injured status, without needing the knowledge of the baseline parameter for the individual. The disclosed approach may also be implemented by a computing device.

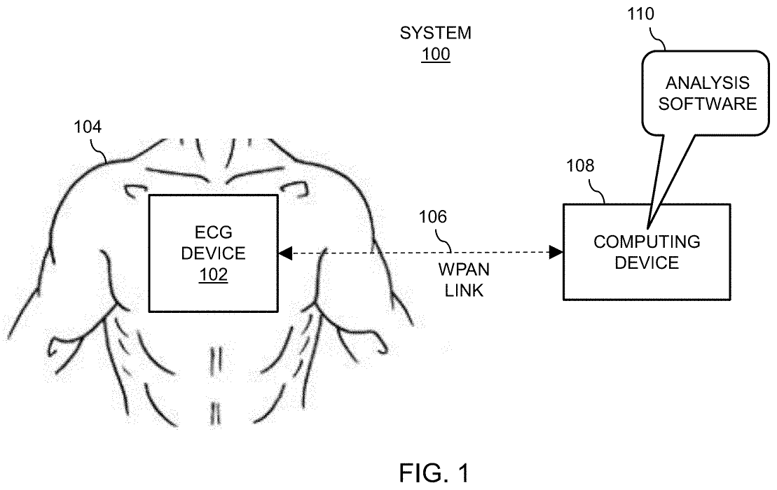

FIG. 1 depicts a block diagram illustrating a system 100 that includes an electrocardiogram (ECG) device 102 worn by a subject 104. The subject 104 may be an athlete, a soldier, a domestic abuse victim, or any other person that recently experienced a head injury. The ECG device 102 includes an electrocardiogram (ECG) sensor and a three-axis accelerometer (not shown in FIG. 1). A wireless personal area network (WPAN) link 106 provides ECG data from the ECG device 102 to a computing device 108. The WPAN link may be a Bluetooth® link. The computing device 108 may be a laptop, a tablet, a smartphone, a smartwatch, or a dedicated medical device. The computing device 108 includes analysis software 110 for diagnosing an mTBI of the subject 104. The system 100 does not require the use of a blood pressure cuff to collect blood pressure data from the subject 104. In other embodiments, ECG device 102 may be replaced with any type of sensor configured to provide beat-to-beat heart interval data.

In other embodiments, the analysis software 110 may be provided via a web portal from a remote server (not shown in FIG. 1). The ECG device 102 may also be configured to communicate with the computing device 108 via a local area network (LAN) such as Wi-Fi network. In still other embodiments, the ECG device 102 may be configured to communicate over a wide area network (WAN). The WAN may be a 3G network, a 4G network, or a 5G network. In this scenario, the computing device may be a remote personal computer (PC) or a remote server. The server may be hosted in a cloud computing environment and may be a virtual server. In other embodiments the ECG device 102 may be integrated with the computing device 108 and the analysis software 110, as a single wearable device to detect an mTBI.

A study using the system 100 of FIG. 1 evaluated biomarkers (i.e. ECG data and accelerometer data) extracted from wearable cardiac sensor data during posture changes (i.e. sequences). The study included 31 subjects previously diagnosed with mTBI within 72 hours of a head injury (i.e. mTBI subjects). The study also included 32 subjects thought not to have suffered an mTBI (i.e. control subjects).

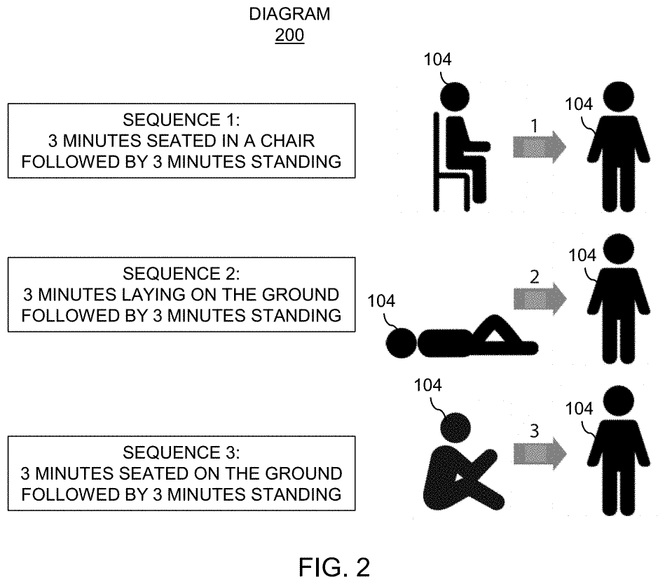

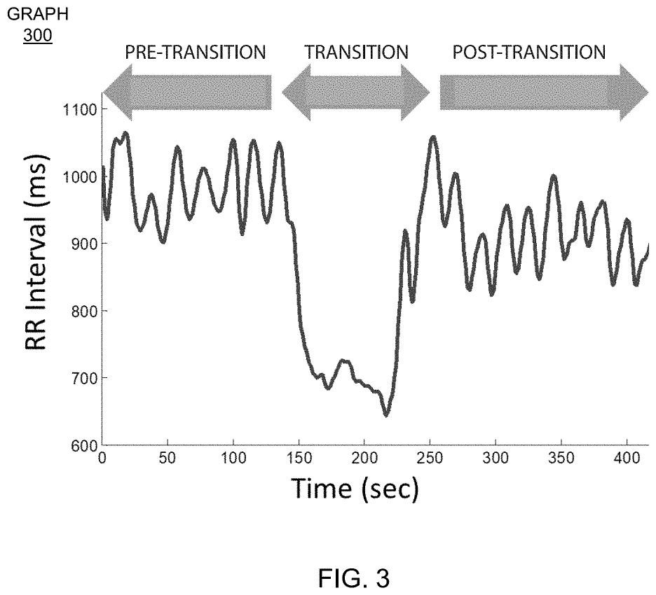

The ECG device 102 of FIG. 1 was used with three difference sequences shown in diagram 200 of FIG. 2 to produce ECG subject transition data. In sequence 1, the subject 104 is seated for approximately three minutes in a chair following by approximately three minutes of standing. In some embodiments for sequence 1, the subject 104 may be seated for a longer and/or shorter timeframe; and may be standing for a longer and/or shorter timeframe. For example, the longer timeframe may be three to five minutes and the shorter time from may be one to three minutes. In sequence 2, the subject 104 lays on the ground for approximately three minutes followed by approximately three minutes of standing. In some embodiments for sequence 2, the subject 104 may lay on the ground for a longer and/or shorter timeframe; and may be standing for a longer or shorter timeframe. In sequence 3, the subject 104 is seated for approximately three minutes on the ground followed by approximately three minutes of standing. In some embodiments for sequence 3, the subject 104 may be seated on the ground for a longer and/or shorter timeframe; and may be standing for a longer or shorter timeframe. FIG. 3 depicts a graph 300 illustrating ECG subject transition data including RR interval versus time of a test subject utilizing at least one sequence of FIG. 2. The graph 300 identifies pre-transition, transition, and post-transition periods after low pass filtering.

For the study, an accurate beat-to-beat (RR) interval was extracted from raw ECG data from ECG device 102 as the starting point for all downstream parameter calculations. The RR interval was characterized in pre-transition, transition, and post-transition periods. Transition timing was determined from accelerometer data provided by the ECG device 102.

The subject biomarkers included differences between pre-transition and post-transition median RR interval values and heart rate variability (HRV) measured in multiple frequency bands. The subject biomarkers also included characteristics of the transition periods including duration, magnitude of the total RR drop, slopes of the RR decrease and recovery, and area of the “u-shaped” RR trough. For purposes of the study, biomarker extraction was automated in Matlab®.

The first step in the analysis of the ECG signal was the identification of R-wave peaks to obtain an accurate R-R interval using the method described in the article titled “Automated respiratory sinus arrhythmia measurement: Demonstration using executive function assessment” by Hegarty-Craver et al. and published Aug. 8, 2017 in Behavior Research Methods of the Psychonomic Society. Beat-to-beat (RR) interval is the difference between successive R-peaks and is the reciprocal of the heart rate.) A low frequency “trend” signal was generated using a third order polynomial low pass filter with 0.04 Hz cutoff. Heart rate variability was captured in two different frequency bands. The low frequency (LF) component of variability was generated by subtraction of the trend signal followed by application of low pass filter with a cutoff of 0.15 Hz. The respiratory sinus arrhythmia (RSA) signal was generated similarly with two filtering stages. First, frequencies below 0.14 Hz were removed by subtracting a low pass filtered version of the signal from the original. Then a 0.14-0.4 Hz bandpass filter was applied.

The original RR interval stream and the three filtered versions were divided into pre-transition, transition, and post-transition segments for each of the three position changes. The exact time of the transition was determined from the sagittal axis acceleration. The raw acceleration data was low pass filtered using a third order polynomial filter with 0.5 Hz cutoff to remove all insignificant movements. The transition time was noted as the time of the maximum of this filtered signal. Using the transition time as a reference, segments were defined as shown in Table I. The 30-second delay in starting the post-transition period was sufficient to exclude the large transient change in RR intervals associated with the transition.

TABLE I

| Segment | Start | Stop |
|---|---|---|
| Pre-Transition | 160 seconds prior | 10 seconds prior |
| Transition | 30 seconds prior | 60 seconds prior |
| Post-Transition | 30 seconds after | 180 seconds after |

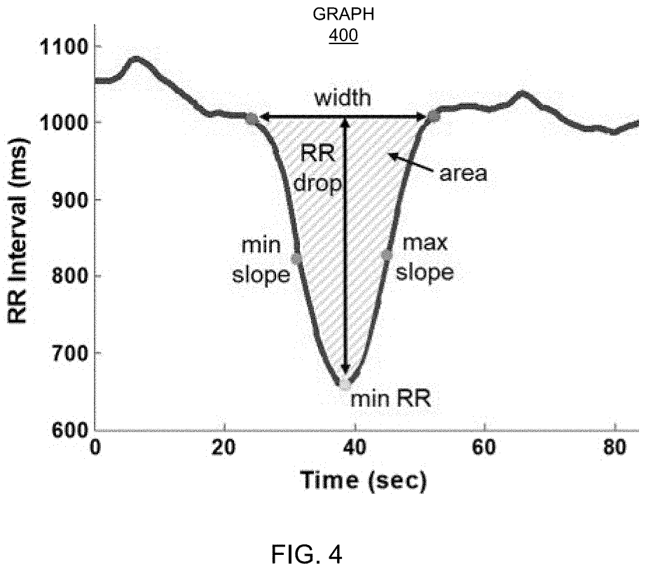

Median values were computed in the pre-transition and post-transition segments for RR intervals, low frequency (LF), respiratory sinus arrhythmia (RSA), and LF/RSA ratio. RSA and LF were quantified using the Porges-Bohrer method described in the article titled “Statistical strategies to quantify respiratory sinus arrhythmia: Are commonly used metrics equivalent?” by Lewis et al. and published Feb. 2, 2012 in ScienceDirect Biological Psychology Volume 89 Issue 2 (pages 349-364). The root mean square (RMS) of the RR trend signal was computed in the post-transition period as a measure of heart rate instability following a position change. In the transition period, the RR trend signal typically showed a u-shape valley with a large decrease and then recovery. The transition period was characterized using the magnitude and location of the minimum RR, maximum slopes on the falling and rising edges, and the width and area of the valley. This is shown in FIG. 4 which depicts a graph 400 illustrating RR intervals versus time utilizing an alternate low pass filtering technique.

The RSA signal was analyzed during the transition period. The RSA gap was defined to be the duration of time that normal fluctuations in the RR signal at the frequency of breathing were temporarily suppressed during the position change.

Lastly, the duration of the physical transition to a different position was defined within the accelerometer data. The three axes of acceleration were combined using sum of squares into a single activity metric. The transition duration was defined as the length of time around the transition that the absolute value of the activity signal remained above a threshold of four standard deviations of the activity level at rest.

Data was analyzed from the 31 mTBI subjects and the 32 control subjects. There were no significant differences between age, height, weight, or BMI between the control and mTBI groups as shown in Table II. The mTBI group had a lower initial heart rate (p<0.01) and higher initial RSA (p<0.01) than the control group.

TABLE II
Table II: Average demographic and baseline
physiological data for the control and mTBIgroups.

| Parameter | Control | mTBI |
|---|---|---|
| Age | 24.0 years | 23.8 years |
| Height | 181.1 in | 186.7 in |
| Weight | 69.2 lbs. | 68.9 lbs. |
| BMI | 26.4 kg/m2 | 27.5 kg/m2 |
| Heart Rate | 68.8 bpm | 61.0 bpm** |
| RSA | 5.97 | 6.69* |
| LF | 6.49 | 6.57 |
| LF/RSA | 1.12 | 1.00 |

*p < .05,
**p < .01

Sequence 3 data was not analyzed but may be included in some embodiments. Parameter values for sequences 1 and 2 are shown in Table III. Only 30 mTBI subjects are included in the sequence 2 analysis as the sensor data was unusable after sequence 1 in one subject.

TABLE III
Table III: Parameter values averaged across
subjects for the control and mTBI groups.

| | Sequence 1 | | Sequence 2 | |
|---|---|---|---|---|
| Parameter | Control | mTBI | Control | mTBI |

Difference Pre- and Post-Transition

| | | | | |
|---|---|---|---|---|
| Mean RR (ms) | 128 | 141 | 237 | 269 |
| Median RSA (ln (ms2)) | 1.38 | 1.18 | 1.81 | 1.72 |
| Median LF (ln (ms2)) | 0.30 | 0.09 | 0.03 | 0.20 |

Transition Metrics

| | | | | |
|---|---|---|---|---|
| Min RR Location (sec) | 10.8 | 12.3** | 11.3 | 12.1 |
| RR Drop (ms) | 279 | 348** | 415 | 512** |
| Min RR Slope (ms/s) | −37.0 | −49.1* | −44.9 | −50.1 |
| Max Slope (ms/s) | 43.0 | 51.1 | 48.2 | 56.2 |
| Max Slope Location (sec) | 11.1 | 12.5* | 10.8 | 12.6** |
| Width of Valley (sec) | 29.8 | 32.4* | 31.7 | 34.7** |
| Area of Valley (sec2) | 3.32 | 5.21** | 5.06 | 7.26** |
| RSA Gap (sec) | 12.7 | 15.2** | 14.1 | 15.7 |
| Transition Duration (sec) | 6.1 | 5.9 | 9.6 | 8.8 |

Post Transition Stability

| | | | | |
|---|---|---|---|---|
| RMS of RR trend (ms) | 768 | 867** | 753 | 859** |

*p < .05,
**p < .01

There was no difference between the groups in the average amount of time that they took to complete the position change (transition duration) for either sequence. Additionally, no significant correlation was found between the transition duration and the cardiac parameters.

Regarding the magnitude of the differences in parameter values in the pre-transition and post-transition segments, there were no significant differences between control and mTBI groups in any of the heart rate or heart rate variability metrics. On average, the mTBI subjects required longer to reach a new steady RR interval magnitude after the transition as indicated by the larger RMS value of the low frequency RR trend signal in the post transition segment. All other significant differences between groups were related to the magnitude and duration of RR interval changes in the transition region. As seen in Table III, there were a few differences between sequence 1 and sequence 2 regarding which parameters showed significant differences between groups.

Logistic regression was used to develop a predictive model for mTBI diagnosis. Sequence 1 and sequence 2 data were combined, omitting the subject without sequence 2 data (n=32 control, 30 mTBI). Missing values were imputed with the average value from all participants.

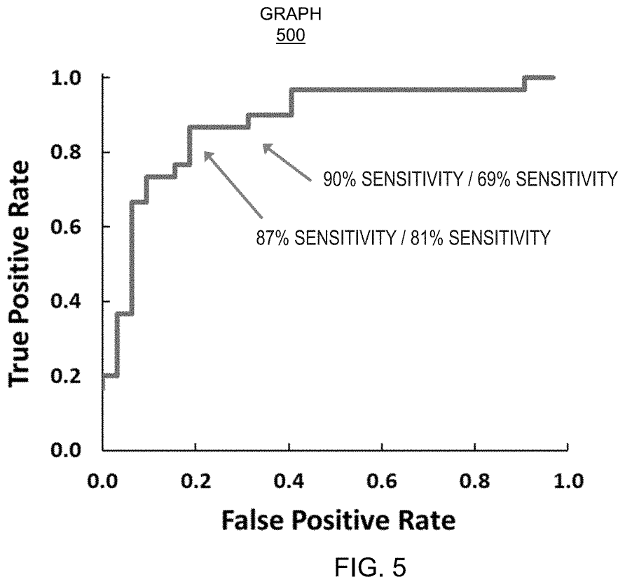

A model was generated using the minimum RR location from sequence 1 and the minimum RR location, the maximum slope location, and the area of the RR valley from sequence 2. This model achieved 90% sensitivity and 69% specificity. The receiver operator characteristic for this combined model is shown in graph 500 of FIG. 5. The area under the curve (AUC) is 0.88. In other embodiments, a predictive model may include different parameters, any sequence individually, or other combinations of sequences.

This study demonstrated that cardiac response to postural change may provide an effective method for diagnosing mTBI. Using a non-invasive, wearable cardiac sensor and a simple posture change procedure, we developed a predictive model for mTBI with 90% sensitivity. Algorithms for biomarker extraction and mTBI prediction can be fully automated in a fieldable diagnostic tool.

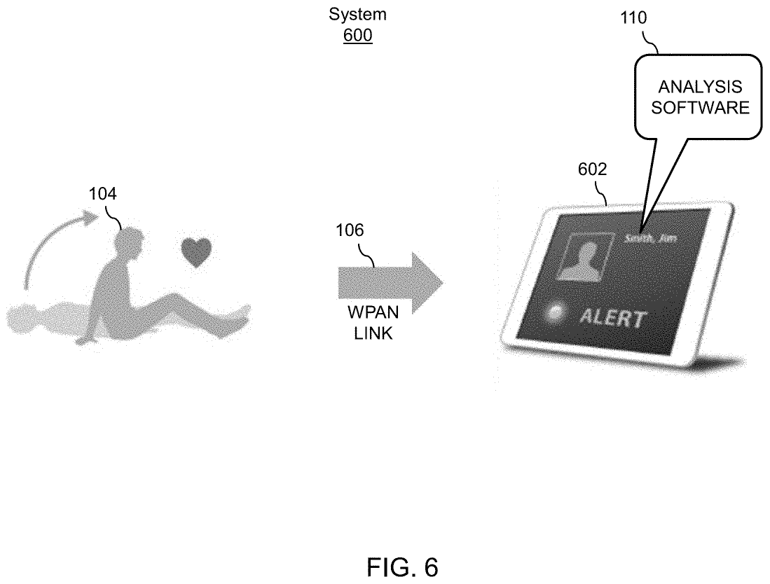

FIG. 6 depicts a block diagram illustrating a system 600 using a smart tablet 602 for objective diagnosis mTBI using a sequence of the subject 104 first laying down and then sitting up in accordance with embodiments of the present disclosure. The smart tablet 602 is configured to provide the analysis software 110 of FIG. 1. In some embodiments, the analysis software 110 may be provided directly as a mobile application that is resident on the smart tablet 602. In other embodiments, the analysis software 110 may be provided by a web portal or a web based application.

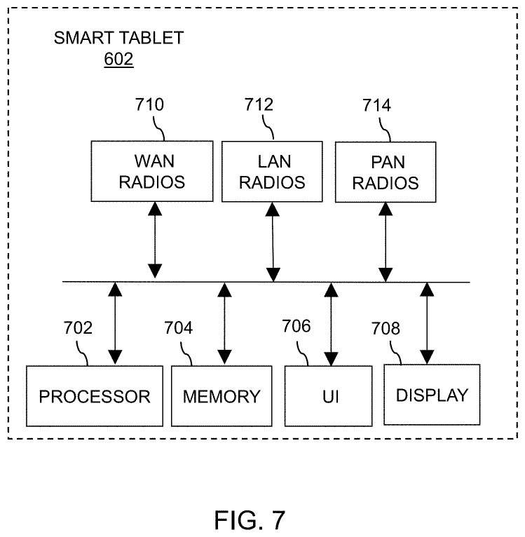

FIG. 7 illustrates a block diagram of the smart tablet 602 in accordance with embodiments of the present disclosure. The smart tablet 602 may include at least a processor 702, a memory 704, a UI 706, a display 708, WAN radios 710, LAN radios 712, and personal area network (PAN) radios 714. In some embodiments the smart tablet 602 may be an iPad®, using iOS® as an operating system (OS). In other embodiments the smart tablet 602 may be a mobile tablet including Android® OS, BlackBerry® OS, Windows Phone® OS, or the like.

In some embodiments, the memory 704 may include a combination of volatile memory (e.g. random access memory) and non-volatile memory (e.g. flash memory). The memory 704 may be partially integrated with the processor 702. The UI 706 and display 708 may be integrated such as a touchpad display. The WAN radios 710 may include 2G, 3G, 4G, and/or 5G technologies. The LAN radios 712 may include Wi-Fi technologies such as 802.11a, 802.11b/g/n, and/or 802.11ac circuitry. The PAN radios 712 may include Bluetooth® technologies, or the like.

Numerous modifications and variations of the present disclosure are possible in view of the above teachings. It is understood that within the scope of the appended claims, the disclosure may be practiced otherwise than as specifically described herein.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. For example, programming languages may include, but are not limited to: Ruby, JavaScript, Java, Python, Ruby, PHP, C, C++, C#, Objective-C, Go, Scala, Swift, Kotlin, OCaml, or the like.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method implemented on a computing device for detecting a mild traumatic brain injury (mTBI) on a subject having recently suffered a head injury, the method comprising:
receiving first electrocardiogram (ECG) data from the subject during a first transition from a first position to a second position; and
determining a probability value of mTBI within the subject based at least partially on the first ECG data during the first transition.

2. The method of claim 1, wherein the first position is at least one of a prone position and a supine position, and the second position is a standing position or prone position.

3. The method of claim 2, wherein the first ECG data is received near real-time from monitoring of an ECG sensor positioned on the subject.

4. The method of claim 3 further comprising receiving first accelerometer data from an accelerometer positioned on the subject, determining the first transition based at least partially on the first accelerometer data, and determining a first baroreflex response associated with the first transition.

5. The method of claim 4, wherein determining a first baroreflex response associated with the first transition includes determining at least one of a magnitude, a rate of change, and a recovery time of beat-to-beat (RR) intervals within the first ECG data.

6. The method of claim 5, wherein determining a first baroreflex response associated with the first transition includes determining a magnitude, a rate of change, and a recovery time of beat-to-beat (RR) intervals within the first ECG data.

7. The method of claim 5, wherein determining a first baroreflex metric response associated with the first transition includes determining a magnitude, a slope, and a time period associated with at least one beat-to-beat (RR) interval within the first ECG data.

8. The method of claim 7 further comprising receiving second ECG data after a second transition of the subject from a sitting position to a standing position and determining a second baroreflex response associated with the second transition.

9. The method of claim 1, wherein the computing device is configured to wirelessly receive the first ECG data.

10. The method of claim 1, wherein the subject is an athlete having recently received a head injury associated with participation in a sport.

11. The method of claim 1, wherein determining the probability value of mTBI within the subject is further based on determining beat-to-beat (RR) trough areas, minimum RR intervals, and maximum recovery slopes associated with the first ECG data.

12. The method of claim 1, wherein the first ECG data is received from a chest positioned ECG sensor.

13. A method implemented on a computing device for detecting a mild traumatic brain injury (mTBI) on a subject having recently suffered a head injury, the method comprising:
receiving first beat-to-beat heart interval data from a sensor configured to monitor a subject during a first transition from a first position to a second position; and
determining a probability value of mTBI within the subject based at least partially on the first beat-to-beat heart interval data during the first transition.

14. The method of claim 13, wherein the sensor is at least one an electrocardiography (ECG) sensor, a photoplethsmography (PPG) sensor, an ultrasonic sensor, and a pressure sensor.

15. The method of claim 13, wherein the computing device is implemented within a diagnostic device comprising the sensor.

16. The method of claim 13, wherein the computing device is implemented within a mobile device configured for receiving the first beat-to-beat heart interval data over at least one of a personal area network (PAN) interface and a local area network (LAN) interface.

17. The method of claim 13, wherein the computing device is implemented within a remote server configured for receiving the first beat-to-beat heart interval data over a wide area network (WAN).

18. A computing device for detecting a mild traumatic brain injury (mTBI) on a subject having recently suffered a head injury, the computing device comprising:
a memory; and
at least one processor configured for:
receiving first beat-to-beat heart interval data from a sensor configured to monitor a subject during a first transition from a first position to a second position; and
determining a probability value of mTBI within the subject based at least partially on the first beat-to-beat heart interval data during the first transition.

19. A method implemented on a computing device for detecting a mild traumatic brain injury (mTBI) on a subject having recently suffered a head injury, the method comprising:
receiving first beat-to-beat heart interval data from a sensor configured to monitor a subject during a baroreflex response within the subject; and
determining a probability value of mTBI within the subject based at least partially on the first beat-to-beat heart interval data during the baroreflex response.

20. The method of claim 19, wherein at least one of a Valsalva maneuver and a cold pressor test produces the baroreflex response of the subject.
